(19) United States
(12) Patent Application Publication
(10) Pub. No.: US 2023/0143116 A1
(43) Pub. Date: May 11, 2023

(54) MEDICAL ASSEMBLIES HAVING SENSOR DEVICES

(71) Applicant: Boston Scientific Medical Device Limited, Galway (IE)

(72) Inventors: Jackie Leung, Richmond Hill (CA); Eduardo Moriyama, Richmond (CA); Gareth Davies, Toronto (CA); Kaylie Lau, Toronto (CA)

(21) Appl. No.: 17/917,640

(22) PCT Filed: Apr. 7, 2021
(86) PCT No.: PCT/IB2021/052907

§ 371 Date: Oct. 7, 2022

Related U.S. Application Data
(60) Provisional application No. 63/007,703, filed on Apr. 9, 2020.

(51) Int. Cl. A61B 5/287; A61B 5/367; A61B 5/06

(52) U.S. Cl. CPC A61B 5/287; A61B 5/367; A61B 5/065; A61B 2562/0209; A61B 2562/043; A61B 2505/05

(57) ABSTRACT

A primary elongated medical assembly is configured to be positioned in, and movable along, a patient. A secondary elongated medical assembly is configured to be positioned in, and movable along, the patient. The primary elongated medical assembly has a primary sensor device. The secondary elongated medical assembly has a secondary sensor device. The primary elongated medical assembly and the secondary elongated medical assembly are each configured to be movable relative to each other; this is done in such a way that the primary sensor device and the secondary sensor device are movable relative to each other.

### TECHNICAL FIELD

This document relates to the technical field of (and is not limited to): (A) medical assemblies having sensor devices (and method therefor); and/or (B) medical assemblies having sensor devices for use with an electro-anatomical mapping system (and method therefor); and/or (C) medical assemblies having sensor devices and an electro-anatomical mapping system (and method therefor).

### BACKGROUND

Known electro-anatomic mapping (EAM) systems are configured to facilitate the mapping of the heart of a patient and/or for electrophysiology procedures, such as cardiac ablation. Cardiac ablation is a procedure that is used to scar small areas in the heart of a patient.

### SUMMARY

It will be appreciated that there exists a need to mitigate (at least in part) at least one problem associated with the existing (known) electro-anatomic mapping (EAM) systems and/or medical assemblies having sensor devices, (also called the existing technology). After much study of, and experimentation with, the existing technology, an understanding (at least in part) of the problem and its solution have been identified (at least in part) and are articulated (at least in part) as follows:

Electro-anatomic mapping (EAM) systems are configured to facilitate mapping and/or cardiac ablation. Cardiac ablation is a procedure to scar or destroy tissue in a heart that's allowing incorrect electrical signals to cause an abnormal heart rhythm, in which diagnostic catheters are threaded through blood vessels to the heart where the catheters are used to map the electrical signals of the heart. Utilization of EAM systems has been shown to reduce fluoroscopic exposure and radiation dose (to patients and/or surgeons, etc.), with less significant effects on procedural duration and success rates. Among the data provided by EAM systems are chamber reconstruction, tagging of important anatomical landmarks and ablation lesions, display of diagnostic and mapping catheters without using fluoroscopy, activation mapping, and voltage (or scar) mapping. EAM systems have specialized features, such as enhanced ability to map unsustained or hemodynamically unstable arrhythmias, ability to display diagnostic as well as mapping catheter positions, and wide compatibility with a variety of catheters. The EAM system may be configured to provide data usable for procedural success (activation mapping, substrate mapping, cardiac geometry), etc. While the EAM system may offer assistance during a procedure, their incorrect or inappropriate application may substantially hamper mapping efforts and procedural success, and should not replace careful interpretation of data and strict adherence to electrophysiologic principles.

Precise transseptal puncture (of the heart of the patient) may be an important factor to ensure accurate Left Atrium (LA) interventions. Electro-anatomic mapping allows the three-dimensional (3D) visualization of a catheter inserted into the heart during pulmonary vein isolation. It may be an advantage to provide at least one or more electrodes positioned on a sheath assembly usable for EAM mapping. It may be desirable to place at least one electrode at a tip portion of a dilator assembly and a combination of sheath electrodes that may be utilized for improved precision transseptal puncture.

For instance, a dilator assembly (a primary medical assembly) may be inserted into a sheath assembly (a secondary medical assembly). The dilator assembly may follow (travel) the same direction in which the sheath assembly is pointed (aligned). For instance, a single dilator electrode, positioned at a dilator tip of the dilator assembly, may protrude from a distal opening of the sheath assembly. The single dilator electrode may be treated as an extension of the sheath electrodes (spaced-apart sensors) of the sheath assembly.

In this manner, the dilator assembly and sheath assembly, as detected or seen by the EAM system, may be treated as, effectively, a single medical assembly by the EAM system. This arrangement may provide improved versatile tracking options for the medical assemblies (such as a combination of a dilator assembly and a sheath assembly) when using the EAM system. It will be appreciated that the EAM system may include equivalent systems, such as a dielectric imaging system, or any type of imaging system, etc.

To mitigate, at least in part, at least one problem associated with the existing technology, there is provided (in accordance with a major aspect) an apparatus. The apparatus includes and is not limited to (comprises) a primary elongated medical assembly configured to be positioned in, and movable along, a patient. The primary elongated medical assembly has a primary sensor device (mounted thereto). The primary elongated medical assembly is configured to be utilized with a secondary elongated medical assembly configured to be positioned in, and movable along, the patient (in which the secondary elongated medical assembly has a secondary sensor device). In accordance with a preferred embodiment, the secondary elongated medical assembly (such as a dilator) is configured to be movable or deliverable within (along) a lumen defined along the primary elongated medical assembly (such as a sheath), and for this case the combination of the primary elongated medical assembly and the secondary elongated medical assembly cooperate (fit, integrate) with each other and a single medical assembly (such as, and not limited to, a combination of a dilator assembly and a sheath assembly, etc.). The primary elongated medical assembly is configured to be movable relative to the secondary elongated medical assembly (this is done in such a way that the primary sensor device and the secondary sensor device are movable relative to each other, that is once the primary elongated medical assembly and the secondary elongated medical assembly, in use, are positioned in, and are movable along, the patient).

To mitigate, at least in part, at least one problem associated with the existing technology, there is provided (in accordance with a major aspect) an apparatus. The apparatus includes and is not limited to (comprises) a primary elongated medical assembly configured to be positioned in, and movable along, a patient. A secondary elongated medical assembly is configured to be positioned in, and movable along, the patient. The primary elongated medical assembly has a primary sensor device (mounted thereto). The secondary elongated medical assembly has a secondary sensor device (mounted thereto). The primary elongated medical assembly and the secondary elongated medical assembly are each configured to be movable relative to each other (this is done in such a way that the primary sensor device and the secondary sensor device are movable relative to each other, that is once the primary elongated medical assembly and the secondary elongated medical assembly, in use, are positioned in, and are movable along, the patient). It will be appreciated that the term “movable relative to each other” includes, for at least at some cases, one assembly that is movable while the other assembly remains stationary, vice versa, or both assemblies are movable at rates of movement (such as speeds) relative to each other.

To mitigate, at least in part, at least one problem associated with the existing technology, there is provided (in accordance with a major aspect) an apparatus. The apparatus includes and is not limited to (comprises) an electro-anatomical mapping system configured to emanate (transmit) an electromagnetic-transmission signal. A primary elongated medical assembly is configured to be positioned in, and movable along, a patient. A secondary elongated medical assembly is configured to be positioned in, and movable along, the patient. The primary elongated medical assembly has a primary sensor device (mounted thereto). The secondary elongated medical assembly has a secondary sensor device (mounted thereto). The primary elongated medical assembly and the secondary elongated medical assembly are each configured to be movable relative to each other (this is done in such a way that the primary sensor device and the secondary sensor device are movable relative to each other, that is once the primary elongated medical assembly and the secondary elongated medical assembly, in use, are positioned in, and are movable along, the patient). The primary sensor device is also configured to receive the electromagnetic-transmission signal emanating from an electro-anatomical mapping system (once the primary elongated medical assembly, in use, is positioned in, and is movable along, the patient). The primary sensor device is also configured to transmit a primary detected signal back to the electro-anatomical mapping system (the electro-anatomical mapping system is also configured to receive the primary detected signal from the primary sensor device). The secondary sensor device is also configured to receive the electromagnetic-transmission signal emanating from the electro-anatomical mapping system (once the secondary elongated medical assembly, in use, is positioned in, and is movable along, the patient). The secondary sensor device is also configured to transmit a secondary detected signal back to the electro-anatomical mapping system (the electro-anatomical mapping system is also configured to receive the secondary detected signal from the secondary sensor device).

To mitigate, at least in part, at least one problem associated with the existing technology, there is provided (in accordance with a major aspect) a method. The method is for utilizing an electro-anatomical mapping system configured to emanate (transmit) an electromagnetic-transmission signal. The method includes and is not limited to (comprises) operational steps (A) to (G), which are identified in no particular order or preferred order of operations. A first operation (A) includes positioning and moving a primary elongated medical assembly in, and along, a patient, in which the primary elongated medical assembly has a primary sensor device (mounted thereto). A second operation (B) includes positioning and moving a secondary elongated medical assembly in, and along, the patient, in which the secondary elongated medical assembly has a secondary sensor device (mounted thereto). A third operation (C) includes moving the primary elongated medical assembly and the secondary elongated medical assembly relative to each other in such a way that the primary sensor device and the secondary sensor device are movable relative to each other, once the primary elongated medical assembly and the secondary elongated medical assembly, in use, are positioned in, and are movable along, the patient. A fourth operation (D) includes utilizing the primary sensor device to receive the electromagnetic-transmission signal emanating from an electro-anatomical mapping system, once the primary elongated medical assembly, in use, is positioned in, and is movable along, the patient. A fifth operation (E) includes utilizing the primary sensor device to transmit a primary detected signal back to the electro-anatomical mapping system, in which the electro-anatomical mapping system is configured to receive the primary detected signal from the primary sensor device. A sixth operation (F) includes utilizing the secondary sensor device to receive the electromagnetic-transmission signal emanating from the electro-anatomical mapping system, once the secondary elongated medical assembly, in use, is positioned in, and is movable along, the patient. A seventh operation (G) includes utilizing the secondary sensor device to transmit a secondary detected signal back to the electro-anatomical mapping system, in which the electro-anatomical mapping system is configured to receive the secondary detected signal from the secondary sensor device.

Other aspects are identified in the claims. Other aspects and features of the non-limiting embodiments may now become apparent to those skilled in the art upon review of the following detailed description of the non-limiting embodiments with the accompanying drawings. This Summary is provided to introduce concepts in simplified form that are further described below in the Detailed Description. This Summary is not intended to identify potentially key features or possible essential features of the disclosed subject matter, and is not intended to describe each disclosed embodiment or every implementation of the disclosed subject matter. Many other novel advantages, features, and relationships will become apparent as this description proceeds. The figures and the description that follow more particularly exemplify illustrative embodiments.

### BRIEF DESCRIPTION OF THE DRAWINGS

The non-limiting embodiments may be more fully appreciated by reference to the following detailed description of the non-limiting embodiments when taken in conjunction with the accompanying drawings, in which:

FIG. 1 depicts a schematic view of an electro-anatomical mapping system; and

FIG. 2, FIG. 3 and FIG. 4 depict side perspective views of a primary elongated medical assembly and a secondary elongated medical assembly for utilization with the electro-anatomical mapping system of FIG. 1.

The drawings are not necessarily to scale and may be illustrated by phantom lines, diagrammatic representations and fragmentary views. In certain instances, details unnecessary for an understanding of the embodiments (and/or details that render other details difficult to perceive) may have been omitted. Corresponding reference characters indicate corresponding components throughout the several figures of the drawings. Elements in the several figures are illustrated for simplicity and clarity and have not been drawn to scale. The dimensions of some of the elements in the figures may be emphasized relative to other elements for facilitating an understanding of the various disclosed embodiments. In addition, common, and well-understood, elements that are useful in commercially feasible embodiments are often not depicted to provide a less obstructed view of the embodiments of the present disclosure.

### LISTING OF REFERENCE NUMERALS USED IN THE DRAWINGS

primary elongated medical assembly 102
primary sensor device 104
sheath assembly 112
secondary tip section 113
lumen 114
spaced-apart sheath electrodes (114A, 114B, 114C, 114D)
sheath tip section 115
secondary elongated medical assembly 202
secondary sensor device 204
dilator assembly 212
secondary tip section 213
dilator electrode assembly 214
dilator tip section 215
patient 800
electro-anatomical mapping system 902
sensor-interface system 904

### DETAILED DESCRIPTION OF THE NON-LIMITING EMBODIMENT(S)

The following detailed description is merely exemplary and is not intended to limit the described embodiments or the application and uses of the described embodiments. As used, the word “exemplary” or “illustrative” means “serving as an example, instance, or illustration.” Any implementation described as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure. The scope of the disclosure is defined by the claims. For the description, the terms “upper,” “lower,” “left,” “rear,” “right,” “front,” “vertical,” “horizontal,” and derivatives thereof shall relate to the examples as oriented in the drawings. There is no intention to be bound by any expressed or implied theory in the preceding Technical Field, Background, Summary or the following detailed description. It is also to be understood that the devices and processes illustrated in the attached drawings, and described in the following specification, are exemplary embodiments (examples), aspects and/or concepts defined in the appended claims. Hence, dimensions and other physical characteristics relating to the embodiments disclosed are not to be considered as limiting, unless the claims expressly state otherwise. It is understood that the phrase “at least one” is equivalent to “a”. The aspects (examples, alterations, modifications, options, variations, embodiments and any equivalent thereof) are described regarding the drawings. It should be understood that the disclosure is limited to the subject matter provided by the claims, and that the disclosure is not limited to the particular aspects depicted and described. It will be appreciated that the scope of the meaning of a device configured to be coupled to an item (that is, to be connected to, to interact with the item, etc.) is to be interpreted as the device being configured to be coupled to the item, either directly or indirectly. Therefore, “configured to” may include the meaning “either directly or indirectly” unless specifically stated otherwise.

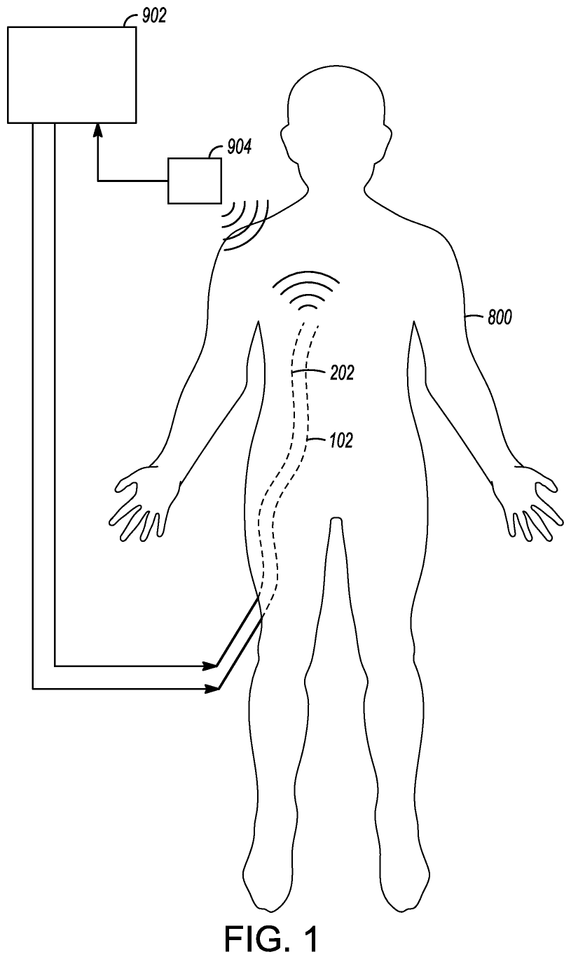

FIG. 1 depicts a schematic view of an electro-anatomical mapping system 902.

Referring to the embodiment as depicted in FIG. 1, an electro-anatomical mapping system 902 is depicted. The electro-anatomical mapping system 902 may include any type of medical signal-measurement system configured to view (detect) any type of medical procedure, a signal analysis system, an electromagnetic system, whether implemented for three-dimensional (3D) viewing and/or two-dimensional (2D) viewing, an electro-anatomic nonfluoroscopic mapping system, a dielectric imaging system, and/or any type of medical-imaging system, etc., and any equivalent thereof. The electro-anatomical mapping system 902 is configured to be electrically connectable (selectively electrically connectable, coupled) to a sensor-interface system 904 configured to transmit an electromagnetic-transmission signal to a primary elongated medical assembly 102 and a secondary elongated medical assembly 202. The primary elongated medical assembly 102 is configured to be positioned in, and movable along, a patient 800. The secondary elongated medical assembly 202 is configured to be positioned in, and movable along, the patient 800. The definition of “electrically connected” includes electro-magnetically connected, magnetically connected, acoustically connected, photonically connected, etc. The sensor-interface system 904 is configured to electromagnetically communicate (interact) with the primary elongated medical assembly 102 and the secondary elongated medical assembly 202.

The electro-anatomical mapping system 902 may be a system configured to provide real-time (near real-time) tracking of the positions of medical sensors (such as electrodes, etc.).

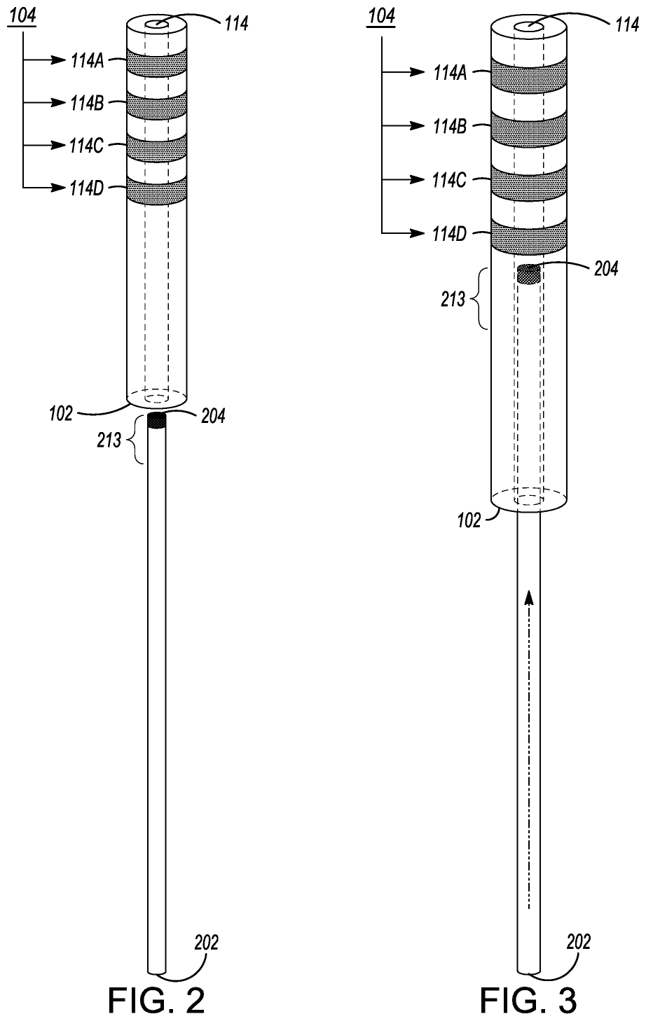

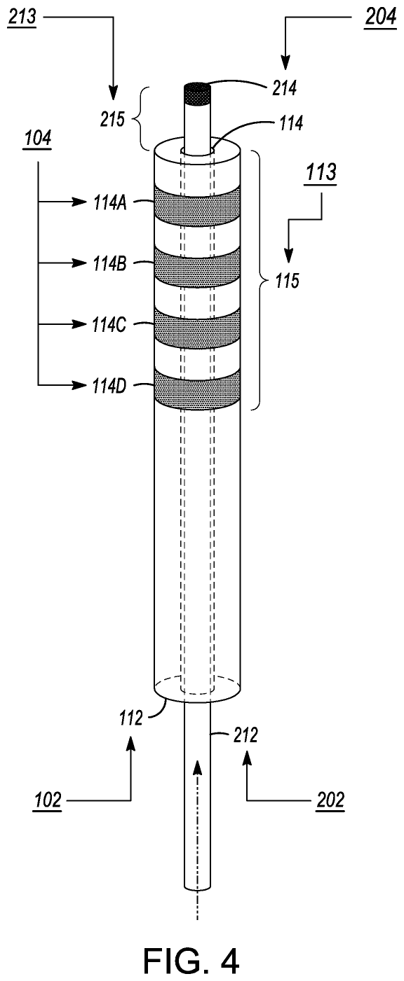

FIG. 2, FIG. 3 and FIG. 4 depict side perspective views of a primary elongated medical assembly 102 and a secondary elongated medical assembly 202 for utilization with the electro-anatomical mapping system 902 of FIG. 1.

Referring to the embodiment as depicted in FIG. 2, the primary elongated medical assembly 102 is positioned adjacent to the secondary elongated medical assembly 202 (in a spaced-apart relationship).

Referring to the embodiment as depicted in FIG. 3, the primary elongated medical assembly 102 is inserted into (at least in part) a proximal end of the secondary elongated medical assembly 202. The proximal end of the secondary elongated medical assembly 202 leads into an interior channel (such as, a lumen 114 or elongated lumen) of the secondary elongated medical assembly 202. The primary elongated medical assembly 102 is moved along toward a distal end of the secondary elongated medical assembly 202 (once or after the primary elongated medical assembly 102 is inserted into the proximal end of the secondary elongated medical assembly 202). The interior channel of the secondary elongated medical assembly 202 extends between the proximal end and the distal end of the secondary elongated medical assembly 202. The proximal end and the distal end (of the secondary elongated medical assembly 202) are in fluid communication with each other. In accordance with a preferred embodiment, the secondary elongated medical assembly 202 is configured to be movable or deliverable within (along) a lumen 114 defined along the primary elongated medical assembly 102, and for this case the combination of the primary elongated medical assembly 102 and the secondary elongated medical assembly 202 cooperate (fit, integrate) with each other and a single medical assembly (such as, and not limited to, a combination of a dilator assembly and a sheath assembly, etc.).

Referring to the embodiment as depicted in FIG. 4, the primary elongated medical assembly 102 is moved through (along) the secondary elongated medical assembly 202 in such a way that a tip portion (such as a secondary tip section 213) of the primary elongated medical assembly 102 exists, and extends from, the distal end of the secondary elongated medical assembly 202.

Referring to the embodiments as depicted in FIG. 2, the primary elongated medical assembly 102 and the secondary elongated medical assembly 202 are (preferably) impermeable by bodily fluids of the patient 800. The primary elongated medical assembly 102 and the secondary elongated medical assembly 202 include bio-compatible material properties suitable for sufficient performance properties (dielectric strength, thermal performance, insulation and corrosion, water and heat resistance) for safe performance to comply with industrial and regulatory safety standards (or compatible for medical usage). Reference is made to the following publication for consideration in the selection of a suitable material: Plastics in Medical Devices: Properties, Requirements, and Applications; 2nd Edition; author: Vinny R. Sastri; hardcover ISBN: 9781455732012; published: 21 Nov. 2013; publisher: Amsterdam [Pays-Bas]: Elsevier/William Andrew, [2014].

Referring to the embodiments as depicted in FIG. 2, FIG. 3 and FIG. 4, the primary elongated medical assembly 102 is configured to be positioned in, and movable along, the patient 800 (which is depicted in FIG. 1). The secondary elongated medical assembly 202 is configured to be positioned in, and movable along, the patient 800 (which is depicted in FIG. 1). The primary elongated medical assembly 102 has a primary sensor device 104 (mounted thereto). The secondary elongated medical assembly 202 has a secondary sensor device 204 (mounted thereto). The primary elongated medical assembly 102 and the secondary elongated medical assembly 202 are each configured to be movable relative to each other. This is done in such a way that the primary sensor device 104 and the secondary sensor device 204 are movable relative to each other (that is, once the primary elongated medical assembly 102 and the secondary elongated medical assembly 202, in use, are positioned in, and are movable along, the patient 800).

Referring to the embodiments as depicted in FIG. 2, FIG. 3 and FIG. 4, the primary sensor device 104 is (preferably) configured to receive an electromagnetic-transmission signal emanating from the electro-anatomical mapping system 902 (which is depicted in FIG. 1); this is done once the primary elongated medical assembly 102, in use, is positioned in, and is movable along, the patient 800. The primary sensor device 104 is configured to transmit a primary detected signal back to the electro-anatomical mapping system 902 (in response to the primary sensor device 104 receiving the electromagnetic-transmission signal from the electro-anatomical mapping system 902). The secondary sensor device 204 is configured to receive the electromagnetic-transmission signal emanating from the electro-anatomical mapping system 902; this is done once the secondary elongated medical assembly 202, in use, is positioned in, and is movable along, the patient 800. The secondary sensor device 204 is configured to transmit a secondary detected signal back to the electro-anatomical mapping system 902 (in response to the secondary sensor device 204 receiving the electromagnetic-transmission signal from the electro-anatomical mapping system 902).

Referring to the embodiments as depicted in FIG. 2, FIG. 3 and FIG. 4, the electro-anatomical mapping system 902 (as depicted in FIG. 1) is (preferably) configured to track relative movements between the primary elongated medical assembly 102 and the secondary elongated medical assembly 202 based on: (A) the primary detected signal of (the primary sensor device 104) that was received by the electro-anatomical mapping system 902, and (B) the secondary detected signal of the secondary sensor device 204 received by the electro-anatomical mapping system 902. In accordance with a preferred embodiment, the electro-anatomical mapping system 902 is configured to track a spatial position of the sensors (electrodes). The display system of the electro-anatomical mapping system 902 may depict movements of the sensors as the assemblies are moved. Therefore the definition of relative movements” is equivalent to “tracking spatial positions”.

Referring to the embodiments as depicted in FIG. 2, FIG. 3 and FIG. 4, the secondary sensor device 204 (preferably) defines a lumen 114 configured to receive, at least in part, the primary sensor device 104 therein and therealong.

Referring to the embodiments as depicted in FIG. 2, FIG. 3 and FIG. 4, the primary elongated medical assembly 102 includes (preferably) a secondary tip section 213. The primary sensor device 104 is mounted (preferably) to the secondary tip section 213.

Referring to the embodiment as depicted in FIG. 2, the secondary elongated medical assembly 202 includes (preferably) a primary tip section 113. The secondary sensor device 204 is mounted to the primary tip section 113.

Referring to the embodiments as depicted in FIG. 2, FIG. 3 and FIG. 4, the electro-anatomical mapping system 902 is (preferably) configured to track relative movements between (A) the secondary tip section 213 of the primary elongated medical assembly 102, and (B) the primary tip section 113 of the secondary elongated medical assembly 202 based on: (i) the primary detected signal (of the primary sensor device 104) that was received by the electro-anatomical mapping system 902, and (ii) the secondary detected signal of the secondary sensor device 204 received by the electro-anatomical mapping system 902.

Referring to the embodiment as depicted in FIG. 4, the primary elongated medical assembly 102 (preferably) includes a dilator assembly 212 having a dilator tip section 215. The primary sensor device 104 (preferably) includes a dilator electrode assembly 214 mounted to the dilator tip section 215. The dilator assembly 212 is configured to be insertable into a lumen (groove, etc.) of a sheath assembly. The dilator assembly 212 may protrude from the tip of the sheath assembly (once it is inserted therein). The dilator assembly 212 is configured to enlarge openings (defined in a biological wall) so the sheath assembly may be movable through (fit through) the enlarged opening, etc. The primary sensor device 104 may include an electrode, etc., and any equivalent thereof. The primary sensor device 104 may include a conductor positioned to a distal surface of the dilator assembly 212. The primary sensor device 104 may be electrically connected (coupled either directly or indirectly) to the electro-anatomical mapping system 902 (as depicted in FIG. 1). The primary sensor device 104 is configured to transmit (nearby) electromagnetic currents to the electro-anatomical mapping system 902 to facilitate tracking of the dilator assembly 212 by the electro-anatomical mapping system 902.

Referring to the embodiment as depicted in FIG. 4, the secondary elongated medical assembly 202 (preferably) includes a sheath assembly 112 having a sheath tip section 115. The sheath assembly 112 defines a lumen 114 configured to receive, at least in part, the dilator assembly 212 therein and therealong. The secondary sensor device 204 (preferably) includes spaced-apart sheath electrodes (114A, 114B, 114C, 114D) positioned along the sheath tip section 115. It will be appreciated that the electrodes positioned or mounted to the primary elongated medical assembly 102 (such as the dilator assembly 212 of FIG. 3) and/or the secondary elongated medical assembly 202 (such as the sheath assembly 112 of FIG. 3) are not limited to the ring-type configuration as depicted in FIG. 2, FIG. 3 and/or FIG. 4. It will be appreciated that the spaced-apart sheath electrodes may include any number of electrodes, and as depicted in the specific embodiment, there are four (4) electrodes (as one possible configuration). The spaced-apart sheath electrodes (114A, 114B, 114C, 114D) are (preferably) positioned on the outer surface of the sheath tip section 115. The sheath assembly 112 may include a catheter, etc., and any equivalent thereof. The sheath assembly 112 is configured to define (provide) a lumen, in which the lumen is configured to guide other medical assemblies into (to) the heart, etc. The secondary sensor device 204 may include an electrode, etc., and any equivalent thereof. The secondary sensor device 204 may include a conductor positioned to a distal surface of the sheath assembly 112. The secondary sensor device 204 may be electrically connected (coupled either directly or indirectly) to the electro-anatomical mapping system 902 (as depicted in FIG. 1). The secondary sensor device 204 is configured to transmit (nearby) electromagnetic currents to the electro-anatomical mapping system 902 to facilitate tracking of the sheath assembly 112 by the electro-anatomical mapping system 902.

Referring to the embodiment as depicted in FIG. 4, the dilator assembly 212 includes the dilator electrode assembly 214 positioned at (on) the tip of the dilator assembly 212, and the sheath assembly 112 includes the spaced-apart sheath electrodes (114A, 114B, 114C, 114D) positioned along a length of the sheath assembly 112 (preferably along the outer surface of the sheath assembly 112). It will be appreciated that a directional vector may be computed by the electro-anatomical mapping system 902 of FIG. 1. The electro-anatomical mapping system 902 treats the sheath assembly 112 and the dilator assembly 212, in combination, as a single medical assembly with multiple sensor devices (such as, five (5) electrodes, the combination of the electrodes of the dilator assembly 212 and the sheath assembly 112). It will be appreciated that the primary sensor device 104 and the secondary sensor device 204 may include any type of sensor, such as an electrode, a coil, an antenna, and/or any device configured to pick up (receive, detect) signals of any kind (such as, electrical signals), and transmit signals (such as, current signals) to the electro-anatomical mapping system 902. The signals may include voltage signals, impedance signals, and electromagnetic field signals, etc., and any equivalent thereof. It will be appreciated that the electro-anatomical mapping system 902 may be configured to independently estimate positions of the dilator assembly 212 and the sheath assembly 112.

Referring to the embodiment as depicted in FIG. 4, the electro-anatomical mapping system 902 (as depicted in FIG. 1) is configured to track relative movements between (A) the dilator tip section 215 of the dilator assembly 212 and (B) the sheath tip section 115 of the sheath assembly 112 based on: (i) the primary detected signal of the dilator electrode assembly 214 received by the electro-anatomical mapping system 902, and (ii) the spaced-apart sheath electrodes (114A, 114B, 114C, 114D) of the sheath assembly 112 received by the electro-anatomical mapping system 902.

Referring to the embodiments as depicted in FIG. 2, FIG. 3 and FIG. 4, there is provided a method of utilizing an electro-anatomical mapping system 902 configured to emanate (transmit) an electromagnetic-transmission signal. It will be appreciated that in accordance with a preferred embodiment, the electro-anatomical mapping system 902 is configured to utilize a quantity of three electromagnetic-transmission signals (signal sources) in order to perform spatial tracking of the sensors of the primary elongated medical assembly 102 and the secondary elongated medical assembly 202. The method has a first operation including positioning and moving a primary elongated medical assembly 102 in, and along, a patient 800 (in which the primary elongated medical assembly 102 has a primary sensor device 104). The method has a second operation including positioning and moving a secondary elongated medical assembly 202 in, and along, the patient 800 (in which the secondary elongated medical assembly 202 has a secondary sensor device 204). The method has a third operation including moving the primary elongated medical assembly 102 and the secondary elongated medical assembly 202 relative to each other; this is done in such a way that the primary sensor device 104 and the secondary sensor device 204 are movable relative to each other (that is, once the primary elongated medical assembly 102 and the secondary elongated medical assembly 202, in use, are positioned in, and are movable along, the patient 800). The method has a fourth operation including utilizing the primary sensor device 104 to receive the electromagnetic-transmission signal emanating from an electro-anatomical mapping system 902 (that is, once the primary elongated medical assembly 102, in use, is positioned in, and is movable along, the patient 800). The method has a fifth operation including utilizing the primary sensor device 104 to transmit a primary detected signal back to the electro-anatomical mapping system 902 (in which the electro-anatomical mapping system 902 is configured to receive the primary detected signal from the primary sensor device 104). The method has a sixth operation including utilizing the secondary sensor device 204 to receive the electromagnetic-transmission signal emanating from the electro-anatomical mapping system 902 (that is, once the secondary elongated medical assembly 202, in use, is positioned in, and is movable along, the patient 800). The method has a seventh operation including utilizing the secondary sensor device 204 to transmit a secondary detected signal back to the electro-anatomical mapping system 902 (in which the electro-anatomical mapping system 902 is configured to receive the secondary detected signal from the secondary sensor device 204).

Regarding the embodiments as depicted in FIG. 2, FIG. 3 and FIG. 4, there is now described the case (this case is not depicted but may be related to FIG. 2 FIG. 3 and FIG. 4), in which the dilator assembly (with a single dilator electrode) is deployed, in a procedure, along with a sheath assembly (not depicted in which the sheath assembly has no electrodes mounted to the sheath assembly), the single dilator electrode may electrically convey the positional information pertaining only to a single spatial point (in space) for a single electrode; and, on this basis the spatial orientation (spatial elongated alignment) of the dilator assembly cannot be known (determined, ascertained, computed) by the electro-anatomical mapping system 902 (of FIG. 1). Therefore, in view of the problem identified above, it may be desirable to have the electro-anatomical mapping system 902 determine (ascertain, compute, display) the spatial elongated alignment of the dilator assembly 212; in this manner, the surgeon may be able utilize this information for improving, at least in part, surgical tactics and/or the outcome of the procedure. Therefore, to mitigate, at least in part, the above identified problem (of not being able to detect the spatial alignment of the dilator assembly 212 having a single electrode), the sheath assembly 112 may be provided with the spaced-apart sheath electrodes (114A, 114B, 114C, 114D) for use with the dilator assembly 212. For instance, the electro-anatomical mapping system 902 includes a set of electrical connections (electrical inputs). The set of electrical connections are configured to be electrically connected (and assigned to or associated with) respective electrodes of the sheath assembly 112 and the dilator assembly 212. The electro-anatomical mapping system 902 is configured to track the spatial position (movements) for each of the electrodes via the information provided by the electrodes to the electro-anatomical mapping system 902 through the set of electrical connections. The dilator electrode assembly 214 (of the dilator electrode assembly 214) may be electrically connected, for electrical continuity, to a first electrical connection of the set of electrical connections (of the-anatomical mapping system 902). The spaced-apart sheath electrodes (114A, 114B, 114C, 114D), of the sheath assembly 112, may be electrically connected, for electrical continuity, to respective electrical connections of the set of electrical connections (of the-anatomical mapping system 902). Each electrode (of the sheath assembly 112 and the dilator assembly 212) may be tracked independently by the electro-anatomical mapping system 902 (after, or once, the electrical connections are made). It will be appreciated that the electrodes of the sheath assembly 112 may provide, at least in part, the spatial alignment information for the dilator assembly 212, while the electrode of the dilator assembly 212 may indicate the spatial position of the dilator tip. The electro-anatomical mapping system 902 may now provide (display, compute, determine) the spatial alignment information for the dilator assembly 212 (via the sheath electrodes of the sheath assembly 112, since the dilator assembly 212 is inserted into the sheath assembly 112), while the dilator electrode of the dilator assembly 212 may indicate the spatial position of the dilator tip. The display of the electro-anatomical mapping system 902 may now depict both types of information to the surgeon during the procedure. The spatial alignment information may include a directional vector and/or a model of the orientation (and potentially curve) of the dilator assembly 212.

The following is offered as further description of the embodiments, in which any one or more of any technical feature (described in the detailed description, the summary and the claims) may be combinable with any other one or more of any technical feature (described in the detailed description, the summary and the claims). It is understood that each claim in the claims section is an open ended claim unless stated otherwise. Unless otherwise specified, relational terms used in these specifications should be construed to include certain tolerances that the person skilled in the art would recognize as providing equivalent functionality. By way of example, the term perpendicular is not necessarily limited to 90.0 degrees, and may include a variation thereof that the person skilled in the art would recognize as providing equivalent functionality for the purposes described for the relevant member or element. Terms such as “about” and “substantially”, in the context of configuration, relate generally to disposition, location, or configuration that are either exact or sufficiently close to the location, disposition, or configuration of the relevant element to preserve operability of the element within the disclosure which does not materially modify the disclosure. Similarly, unless specifically made clear from its context, numerical values should be construed to include certain tolerances that the person skilled in the art would recognize as having negligible importance as they do not materially change the operability of the disclosure. It will be appreciated that the description and/or drawings identify and describe embodiments of the apparatus (either explicitly or inherently). The apparatus may include any suitable combination and/or permutation of the technical features as identified in the detailed description, as may be required and/or desired to suit a particular technical purpose and/or technical function. It will be appreciated that, where possible and suitable, any one or more of the technical features of the apparatus may be combined with any other one or more of the technical features of the apparatus (in any combination and/or permutation). It will be appreciated that persons skilled in the art would know that the technical features of each embodiment may be deployed (where possible) in other embodiments even if not expressly stated as such above. It will be appreciated that persons skilled in the art would know that other options may be possible for the configuration of the components of the apparatus to adjust to manufacturing requirements and still remain within the scope as described in at least one or more of the claims. This written description provides embodiments, including the best mode, and also enables the person skilled in the art to make and use the embodiments. The patentable scope may be defined by the claims. The written description and/or drawings may help to understand the scope of the claims. It is believed that all the crucial aspects of the disclosed subject matter have been provided in this document. It is understood, for this document, that the word “includes” is equivalent to the word “comprising” in that both words are used to signify an open-ended listing of assemblies, components, parts, etc. The term “comprising”, which is synonymous with the terms “including,” “containing,” or “characterized by,” is inclusive or open-ended and does not exclude additional, unrecited elements or method steps. Comprising (comprised of) is an “open” phrase and allows coverage of technologies that employ additional, unrecited elements. When used in a claim, the word “comprising” is the transitory verb (transitional term) that separates the preamble of the claim from the technical features of the disclosure. The foregoing has outlined the non-limiting embodiments (examples). The description is made for particular non-limiting embodiments (examples). It is understood that the non-limiting embodiments are merely illustrative as examples.

What is claimed is:

1. An apparatus, comprising:
a primary elongated medical assembly configured to be positioned in, and movable along, a patient; and
the primary elongated medical assembly having a primary sensor device; and
the primary elongated medical assembly configured to be utilized with a secondary elongated medical assembly configured to be positioned in, and movable along, the patient, and in which the secondary elongated medical assembly has a secondary sensor device; and
the primary elongated medical assembly configured to be movable relative to the secondary elongated medical assembly in such a way that the primary sensor device and the secondary sensor device are movable relative to each other, once the primary elongated medical assembly and the secondary elongated medical assembly, in use, are positioned in, and are movable along, the patient.

2. An apparatus, comprising:
a primary elongated medical assembly configured to be positioned in, and movable along, a patient; and
a secondary elongated medical assembly configured to be positioned in, and movable along, the patient; and
the primary elongated medical assembly having a primary sensor device; and
the secondary elongated medical assembly having a secondary sensor device; and
the primary elongated medical assembly and the secondary elongated medical assembly each configured to be movable relative to each other in such a way that the primary sensor device and the secondary sensor device are movable relative to each other, once the primary elongated medical assembly and the secondary elongated medical assembly, in use, are positioned in, and are movable along, the patient.

3. The apparatus of claim 2, wherein:
the primary sensor device is configured to receive an electromagnetic-transmission signal emanating from an electro-anatomical mapping system, once the primary elongated medical assembly, in use, is positioned in, and is movable along, the patient; and
the primary sensor device is also configured to transmit a primary detected signal back to the electro-anatomical mapping system, and the electro-anatomical mapping system is also configured to receive the primary detected signal from the primary sensor device; and
the secondary sensor device is configured to receive the electromagnetic-transmission signal emanating from the electro-anatomical mapping system, once the secondary elongated medical assembly, in use, is positioned in, and is movable along, the patient; and
the secondary sensor device is also configured to transmit a secondary detected signal back to the electro-anatomical mapping system, and the electro-anatomical mapping system is also configured to receive the secondary detected signal from the secondary sensor device.

4. The apparatus of claim 3, wherein:
the electro-anatomical mapping system is configured to track relative movements between the primary elongated medical assembly and the secondary elongated medical assembly based on:
the primary detected signal, of the primary sensor device, that was received by the electro-anatomical mapping system; and
the secondary detected signal of the secondary sensor device received by the electro-anatomical mapping system.

5. The apparatus of claim 3, wherein:
the secondary sensor device defines a lumen configured to receive, at least in part, the primary sensor device therein and therealong.

6. The apparatus of claim 3, wherein:
the primary elongated medical assembly includes a secondary tip section; and
the primary sensor device is mounted to the secondary tip section.

7. The apparatus of claim 6, wherein:
the secondary elongated medical assembly includes a primary tip section; and
the secondary sensor device is mounted to the primary tip section.

8. The apparatus of claim 7, wherein:
the electro-anatomical mapping system is configured to track relative movements between the secondary tip section of the primary elongated medical assembly and the primary tip section of the secondary elongated medical assembly based on:
the primary detected signal, of the primary sensor device, that was received by the electro-anatomical mapping system; and
the secondary detected signal of the secondary sensor device received by the electro-anatomical mapping system.

9. The apparatus of claim 2, wherein:
the primary elongated medical assembly includes a dilator assembly having a dilator tip section.

10. The apparatus of claim 9, wherein:
the primary sensor device includes a dilator electrode assembly mounted to the dilator tip section.

11. The apparatus of claim 10, wherein:
the secondary elongated medical assembly includes:
a sheath assembly having a sheath tip section, and defining a lumen configured to receive, at least in part, the dilator assembly therein and therealong.

12. The apparatus of claim 11, wherein:
the secondary sensor device includes:
spaced-apart sheath electrodes (A, B, C, D) positioned along the sheath tip section.

13. The apparatus of claim 12, wherein:
the primary sensor device IS configured to receive an electromagnetic-transmission signal emanating from an electro-anatomical mapping system, once the primary elongated medical assembly, in use, is positioned in, and is movable along, the patient; and
the secondary sensor device is configured to receive the electromagnetic-transmission signal emanating from the electro-anatomical mapping system, once the secondary elongated medical assembly, in use, is positioned in, and is movable along, the patient; and
the electro-anatomical mapping system is configured to track relative movements between the dilator tip section of the dilator assembly and the sheath tip section of the sheath assembly.

14. The apparatus of claim 12, wherein:
the primary sensor device is also configured to transmit a primary detected signal back to an electro-anatomical mapping system, and the electro-anatomical mapping system is also configured to receive the primary detected signal from the primary sensor device; and
the secondary sensor device is also configured to transmit a secondary detected signal back to the electro-anatomical mapping system, and the electro-anatomical mapping system is also configured to receive the secondary detected signal from the secondary sensor device; and
the electro-anatomical mapping system is configured to track relative movements between the dilator tip section of the dilator assembly and the sheath tip section of the sheath assembly based on:
the primary detected signal of the dilator electrode assembly received by the electro-anatomical mapping system; and
spaced-apart sheath electrodes (A, B, C, D) of the sheath assembly received by the electro-anatomical mapping system.

15. The apparatus in accordance with claim 1, wherein:
the secondary elongated medical assembly is configured to be movable or deliverable along a lumen defined along the primary elongated medical assembly.

16. An apparatus, comprising:
an electro-anatomical mapping system configured to emanate an electromagnetic-transmission signal; and
a primary elongated medical assembly configured to be positioned in, and movable along, a patient; and
a secondary elongated medical assembly configured to be positioned in, and movable along, the patient; and
the primary elongated medical assembly having a primary sensor device; and
the secondary elongated medical assembly having a secondary sensor device; and
the primary elongated medical assembly and the secondary elongated medical assembly each configured to be movable relative to each other in such a way that the primary sensor device and the secondary sensor device are movable relative to each other, once the primary elongated medical assembly and the secondary elongated medical assembly, in use, are positioned in, and are movable along, the patient.

17. The apparatus of claim 16, wherein:
the primary sensor device is also configured to receive the electromagnetic-transmission signal emanating from an electro-anatomical mapping system, once the primary elongated medical assembly, in use, is positioned in, and is movable along, the patient.

18. The apparatus of claim 17, wherein:
the primary sensor device is also configured to transmit a primary detected signal back to the electro-anatomical mapping system, and the electro-anatomical mapping system is also configured to receive the primary detected signal from the primary sensor device.

19. The apparatus of claim 18, wherein:
the secondary sensor device is also configured to receive the electromagnetic-transmission signal emanating from the electro-anatomical mapping system, once the secondary elongated medical assembly, in use, is positioned in, and is movable along, the patient.

20. The apparatus of claim 19, wherein:
the secondary sensor device is also configured to transmit a secondary detected signal back to the electro-anatomical mapping system, and the electro-anatomical mapping system is also configured to receive the secondary detected signal from the secondary sensor device.

21-22. (canceled)
